(19) United States
(12) Patent Application Publication
(10) Pub. No.: US 2025/0191787 A1
(43) Pub. Date: Jun. 12, 2025

(54) INFORMATION PROCESSING APPARATUS, INFORMATION PROCESSING TERMINAL, INFORMATION PROCESSING METHOD, AND PROGRAM

(71) Applicant: SONY GROUP CORPORATION, TOKYO (JP)

(72) Inventors: MASAHITO YAMANE, TOKYO (JP); MASAYA TAKEMOTO, TOKYO (JP)

(21) Appl. No.: 18/840,934

(22) PCT Filed: Feb. 15, 2023
(86) PCT No.: PCT/JP2023/005122

§ 371 Date: Aug. 23, 2024

(30) Foreign Application Priority Data
Mar. 3, 2022 (JP) 2022-032240

(51) Int. Cl. G16H 80/00; G06V 40/16; H04N 7/18

(52) U.S. Cl. CPC G16H 80/00; G06V 40/161; G06V 40/168; G06V 40/174; H04N 7/181; G06V 2201/03; G06V 2201/10

(57) ABSTRACT

The present technology relates to an information processing apparatus, an information processing terminal, an information processing method, and a program each adapted to be capable of appropriately selecting a mode for transmitting information regarding an expression. An information processing apparatus according to a first aspect of the present technology sets, in a case where an operation video requested from a remote terminal is a face video possibly showing a patient's face, a transmission mode on the basis of a transmission setting defined for the remote terminal to which the face video is transmitted, transmits information on a feature point of the patient's face extracted from the face video to the remote terminal in a case where a first transmission mode is set, and transmits the compressed face video to the remote terminal in a case where a second transmission mode is set. The present technology is applicable to a remote operation system for instructing an operator in an operating room on how to conduct an operation from a remote location.

### TECHNICAL FIELD

The present technology relates to an information processing apparatus, an information processing terminal, an information processing method, and a program. The present technology particularly relates to an information processing apparatus, an information processing terminal, an information processing method, and a program each adapted to be capable of appropriately selecting a mode for transmitting information regarding an expression.

### BACKGROUND ART

A remote operation has been proposed, which refers to an operation to be conducted by a medical staff in an operating room in accordance with instructions and advice given by a doctor viewing a real-time video showing the operation, from a remote location. In a case where the real-time video shows a patient's face corresponding to personal information, it is necessary to determine whether to share information on the patient's face with the doctor who is at the remote location, that is, who is outside a hospital from the viewpoint of privacy protection, depending on circumstances.

Patent Document 1 discloses a technique for generating an operation video showing another person's face with which a patient's face is replaced.

### CITATION LIST

### Patent Document

Patent Document 1: WO 2019/244896 A

### SUMMARY OF THE INVENTION

### Problems to be Solved by the Invention

In transmitting and receiving an operation video, it is also required to transmit and receive an appropriate video in response to various situations such as a communication state, while taking privacy protection into consideration.

For example, in some cases, a video showing an operative field is preferentially transmitted, and a video showing a patient's face is not transmitted, depending on a communication zone between a remote location and an operating room. However, a change in a patient's face is important information for making a diagnosis and recognizing a problem about the progress of an operation. Therefore, a system has been desired, which allows a doctor to grasp the change in the patient's face at a remote location.

The present technology has been made in view of the circumstances described above and is adapted to be capable of appropriately selecting a mode for transmitting information regarding an expression.

### Solutions to Problems

An information processing apparatus according to a first aspect of the present technology includes: a transmission mode setting unit that sets, in a case where an operation video requested from a remote terminal is a face video possibly showing a patient's face, a transmission mode on the basis of a transmission setting defined for the remote terminal to which the face video is transmitted; and a transmission control unit that transmits information on a feature point of the patient's face extracted from the face video to the remote terminal in a case where a first transmission mode is set, and transmits the compressed face video to the remote terminal in a case where a second transmission mode is set.

An information processing terminal according to a second aspect of the present technology includes: a transmitted data acquisition unit that sets, in a case where an operation video of which transmission is requested is a face video possibly showing a patient's face, a transmission mode on the basis of a transmission setting defined for a destination to which the face video is transmitted, transmits information on a feature point of the patient's face extracted from the face video in a case where a first transmission mode is set, and acquires information transmitted from an information processing apparatus configured to transmit the compressed face video, in a case where a second transmission mode is set; and a display control unit that displays information regarding a patient's expression on the basis of the acquired information.

According to the first aspect of the present technology, in the case where the operation video requested from the remote terminal is the face video possibly showing the patient's face, the transmission mode is set on the basis of the transmission setting defined for the remote terminal to which the face video is transmitted, the information on the feature point of the patient's face extracted from the face video is transmitted to the remote terminal in the case where the first transmission mode is set, and the compressed face video is transmitted to the remote terminal in the case where the second transmission mode is set.

According to the second aspect of the present technology, in the case where the operation video of which transmission is requested is the face video possibly showing the patient's face, the transmission mode is set on the basis of the transmission setting defined for the destination to which the face video is transmitted, the information on the feature point of the patient's face extracted from the face video is transmitted in the case where the first transmission mode is set, the information transmitted from the information processing apparatus configured to transmit the compressed face video is acquired in the case where the second transmission mode is set, and the information regarding the patient's expression is displayed on the basis of the acquired information.

### BRIEF DESCRIPTION OF DRAWINGS

FIG. 1 is a diagram illustrating an exemplary configuration of an information processing system according to an embodiment of the present technology.

FIG. 2 is a diagram illustrating exemplary display in a remote terminal.

FIG. 3 is a diagram illustrating an exemplary transmission setting.

FIG. 4 is a diagram illustrating an exemplary configuration of an operating room system.

FIG. 5 is a block diagram illustrating an exemplary functional configuration of an operating room server constituting a part of the operating room system.

FIG. 6 is a diagram illustrating an exemplary feature point extracted from a face.

FIG. 7 is a flowchart illustrating processing to be performed by the operating room server.

FIG. 8 is a block diagram illustrating an exemplary functional configuration of the remote terminal.

FIG. 9 is a flowchart illustrating processing to be performed by the remote terminal.

FIG. 10 is a diagram illustrating exemplary another instrument that makes a face video determination.

FIG. 11 is a diagram illustrating an exemplary IP converter that performs operation video processing.

FIG. 12 is a diagram illustrating another exemplary configuration of the information processing system according to the embodiment of the present technology.

FIG. 13 is a block diagram illustrating an exemplary hardware configuration of a computer.

### MODE FOR CARRYING OUT THE INVENTION

A mode for carrying out the present technology will be described below. The description is given in the following order.
1. Overview of the present technology
2. Configuration and behavior of operating room system
3. Configuration and behavior of remote terminal
4. Modifications

### Overview of the Present Technology

### <Configuration of Information Processing System>

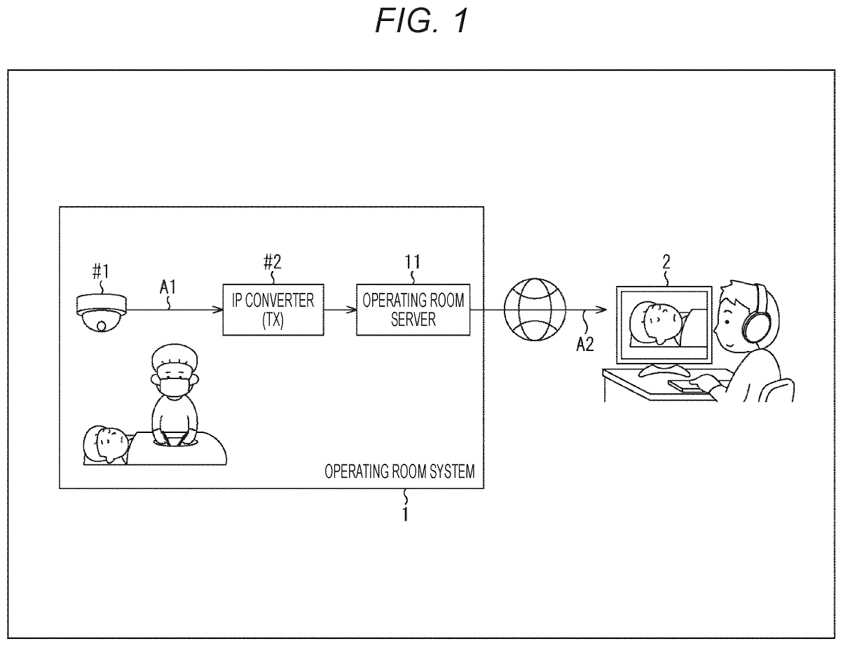

FIG. 1 is a diagram illustrating an exemplary configuration of an information processing system according to an embodiment of the present technology.

The information processing system according to the embodiment of the present technology includes an operating room system 1 and a remote terminal 2. The information processing system illustrated in FIG. 1 is a system for a remote operation that refers to an operation to be conducted by an operator who is in an operating room, in accordance with guidance about how to conduct the operation by a doctor who is at a remote location (a user of the remote terminal 2) and acts as a supervisory doctor, for example.

The operating room system 1 is introduced in a medical facility having an operating room, such as a hospital. As will be described later, the operating room system 1 includes a plurality of cameras such as a camera that images an operative field, a camera that images a patient's face, and a camera that images how things are at an operating room. Persons such as the operator and the patient illustrated in FIG. 1 do not constitute the operating room system 1.

The remote terminal 2 is a terminal located apart from the operating room. The remote terminal 2 is constituted of a PC, a tablet terminal, or the like. For example, the doctor who is at the remote location is a user of the remote terminal 2 and manipulates the remote terminal 2. The remote terminal 2 may be used at various locations, such as a user's home and a room in a medical facility in which the operating room system 1 is introduced, as long as the remote terminal 2 is used at a location far from the operating room. The operating room system 1 and the remote terminal 2 communicate with each other via the Internet.

The operating room system 1 and the remote terminal 2 may communicate with each other under the control by a server on the Internet. Note that although FIG. 1 illustrates only one remote terminal 2, multiple remote terminals 2 are connected to the operating room system 1 via the Internet.

In the information processing system having the configuration described above, a camera #1 constituting a part of the operating room system 1 images a video showing how things are during an operation and transmits the video to the remote terminal 2. For example, the multiple cameras of the operating room system 1 respectively image different operation videos. Of the multiple operation videos, an operation video requested from the user of the remote terminal 2 is transmitted to the remote terminal 2.

The camera #1 illustrated in FIG. 1 is a camera placed on the ceiling of the operating room. The operation video imaged by the camera #1 is output to an IP converter #2 as indicated by an arrow A1 in FIG. 1.

The IP converter #2 subjects the operation video supplied from the camera #1 to IP conversion, and outputs the operation video subjected to IP conversion to the operating room server 11.

The operating room server 11 determines whether or not the operation video imaged by the camera #1 and supplied from the IP converter #2 is a video possibly showing the patient's face. The determination on the operation video may be made by the camera #1 or the IP converter #2 as will be described later. Hereinafter, the operation video possibly showing the patient's image is appropriately referred to as a face video.

In a case where the operation video imaged by the camera #1 is determined as the face video and a request to transmit the operation video (the face video) imaged by the camera #1 is made by the remote terminal 2, the operating room server 11 sets a transmission mode for the face video on the basis of a transmission setting defined for the remote terminal 2 to which the face video is transmitted. The transmission setting is information for prescribing how to set a transmission mode. For example, a transmission setting is prepared for each remote terminal 2.

As the transmission mode for a face video, for example, a first transmission mode and a second transmission mode are prepared.

The first transmission mode is a mode for transmitting information on a feature point of a patient's face extracted from a face video.

The second transmission mode is a mode for transmitting a compressed face video. For example, a 4K-resolution face video imaged by the camera #1 is converted to a FHD (2K)-resolution face video (i.e., the resolution is reduced), and the face video obtained by the conversion is transmitted as a compressed face video.

The face video is transmitted in accordance with the transmission mode set by the operating room server 11. In a case where the first transmission mode is set, the information on the feature point of the patient's face is transmitted to the remote terminal 2 as indicated by an arrow A2. In a case where the second transmission mode is set, the compressed face video is transmitted to the remote terminal 2 as indicated by the arrow A2.

The remote terminal 2 that has received the information transmitted in the first transmission mode or the second transmission mode displays a screen including information regarding a patient's expression on a display on the basis of the information transmitted from the operating room server 11.

### <Exemplary Display in Remote Terminal 2>

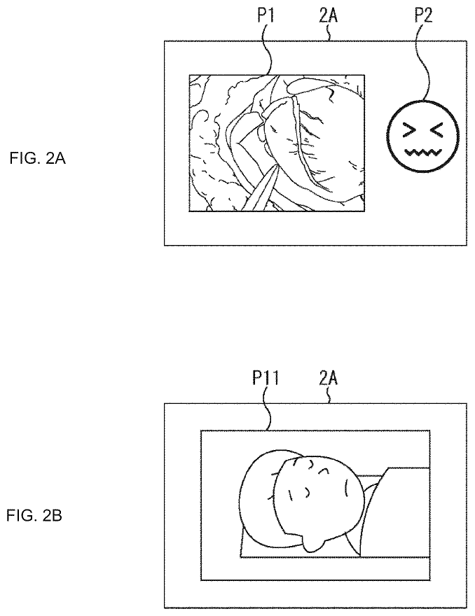

FIG. 2 is a diagram illustrating exemplary display in the remote terminal 2.

As illustrated in FIG. 2, the remote terminal 2 includes the display 2A that displays the information transmitted from the operating room server 11, in various forms.

A screen A illustrated in FIG. 2 is a screen displayed on the basis of the information transmitted in the first transmission mode. The screen A illustrated in FIG. 2 displays an operative field video P1 and an avatar image P2 representing the patient's expression. The expression represented by the avatar image P2 changes on the basis of the information on the feature point of the patient's face, the information being transmitted from the operating room server 11. In this example, the avatar image P2 corresponds to the information regarding the patient's expression.

As described above, in the case where the face video is transmitted in the first transmission mode, the remote terminal 2 presents the patient's expression to the user, using the avatar image P2. In a case where an image of the patient's face has already been provided on the remote terminal 2 side, the patient's expression may be presented to the user by changing the image of the patient's face, which has already been provided, in accordance with the information on the feature point of the face.

A screen B illustrated in FIG. 2 is a screen displayed on the basis of the information transmitted in the second transmission mode. The screen B illustrated in FIG. 2 displays a video P11 obtained by decompressing the compressed face video (i.e., by increasing the resolution). The video P11 shows the patient's face.

As described above, in the case where the face video is transmitted in the second transmission mode, the remote terminal 2 presents the patient's expression to the user by decompressing the compressed face video transmitted from the operating room server 11 and displaying the face video obtained by the decompression.

The user of the remote terminal 2 is able to confirm a change in the patient's face and to give instructions to the operator and the like in the operating room in real-time while viewing the video displayed on the display 2A.

### <Transmission Setting>

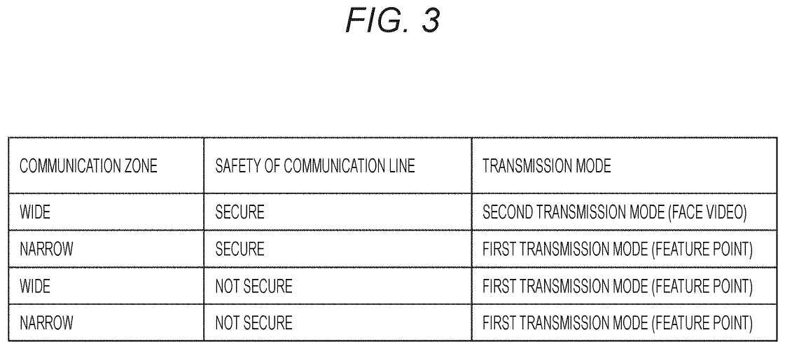

FIG. 3 is a diagram illustrating an exemplary transmission setting.

As illustrated in FIG. 3, the transmission setting indicates a communication zone between the operating room system 1 and the remote terminal 2 and a transmission mode to be set, of the first transmission mode and the second transmission mode, in accordance with the security of a communication line.

As can be seen from the second row of FIG. 3, in a case where the communication zone is wide and the communication line is secure, the second transmission mode is set as the transmission mode.

In the case where the communication zone is wide, a value such as a transmission speed indicating the state of the communication zone is larger than a predetermined threshold value, for example. In contrast, in a case where the communication zone is narrow, the value such as the transmission speed indicating the state of the communication zone is smaller than the predetermined threshold value.

Furthermore, in the case where the security of the communication line is secure, the remote terminal 2 to which the face video is transmitted is a terminal managed by the hospital (a hospital-aligned terminal), for example. A connection between the operating room system 1 and the remote terminal 2 as the hospital-aligned terminal is established by a virtual private network (VPN). In a case where the remote terminal 2 to which the face video is transmitted is different from a hospital-aligned terminal, the security of the communication line is not secure.

As can be seen from the third row of FIG. 3, in a case where the communication zone is narrow and the communication line is secure, the first transmission mode is set as the transmission mode.

As can be seen from the fourth row, in a case where the communication zone is wide and the communication line is not secure, the first transmission mode is set as the transmission mode.

As can be seen from the fifth row, in a case where the communication zone is narrow and the communication line is not secure, the first transmission mode is set as the transmission mode.

The threshold value as a reference for the communication zone may be set in advance or may be set by the user.

Other conditions such as the presence/absence of patient's consent may be prescribed by the transmission setting, in addition to the communication zone and the security of the communication line. That is, the content of the transmission setting can be prescribed by at least one of the state of the communication zone between the operating room system 1 and the remote terminal 2, the security of the communication line, or the presence/absence of the patient's consent.

As described above, in the information processing system illustrated in FIG. 1, in the case where the imaged operation video possibly shows the patient's face, the face video is transmitted in accordance with the transmission mode according to the state of the communication line or the like.

Therefore, it is possible to appropriately select the mode for transmitting the information regarding the patient's expression. For example, it is possible to accurately send the information regarding the patient's expression to the doctor who is at the remote location without transmitting the patient's personal information, by sending only the information on the feature point of the patient's face rather than the face video showing the patient's face.

### <<Configuration and Behavior of Operating Room System>

### <Configuration of Operating Room System 1>

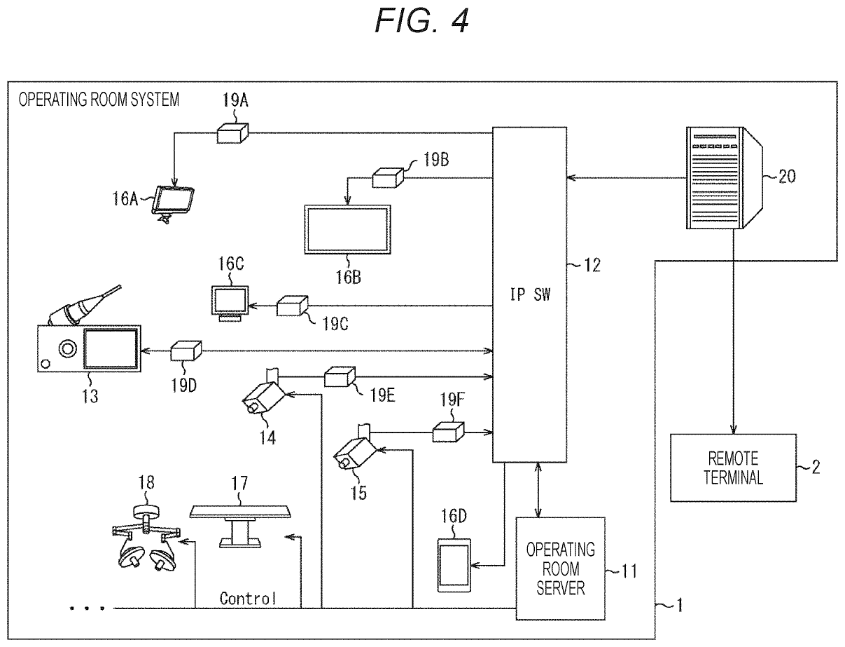

FIG. 4 is a diagram illustrating an exemplary configuration of the operating room system 1 illustrated in FIG. 1.

In the operating room system 1, a group of devices placed in the operating room are connected in cooperation with each other via the operating room server 11 and an IP switch (SW) 12. The operating room system 1 includes an Internet protocol (IP) network capable of transmitting and receiving a 4K/8K image, and an input/output image and control information for each instrument are transmitted and received via the IP network.

Various devices are placed in the operating room. FIG. 4 illustrates, for example, a group of various devices for endoscopic surgery (a device group 13), a ceiling camera 14 that images the hands of the operator, an operative field camera 15 that images how things are at the entire operating room, display devices 16A to 16D, a patient bed 17, and a light 18. Both the ceiling camera 14 and the operative field camera 15 are mounted to the ceiling of the operating room. The device group 13 may include, in addition to an illustrated endoscope, various medical instruments for acquiring images, such as a master-slave endoscopic surgical robot and an X-ray machine.

The display devices 16A to 16C, the device group 13, the ceiling camera 14, and the operative field camera 15 are connected to the IP SW 12 via IP converters 19A to 19F, respectively. Hereinafter, the IP converters 19A to 19F and the like are simply referred to as the IP converter(s) 19 unless they are distinguished from each other.

Each of the IP converters 19D, 19E, and 19F is an input source-side (camera-side) IP converter 19 to which an image is input. The IP converters 19D, 19E, and 19F subject images from individual medical imaging devices (e.g., an endoscope, a surgical microscope, an X-ray machine, an operative field camera, a pathologic image capture device) to IP conversion, and transmit the resultant images onto the network. For example, the endoscopic camera in the device group 13, the ceiling camera 14, and the operative field camera 15 each correspond to the camera #1 illustrated in FIG. 1, and the IP converters 19D, 19E, and 19F each correspond to the IP converter #2 illustrated in FIG. 1.

Each of the IP converters 19A to 19C is an image output-side (monitor-side) IP converter 19 from which an image is output. The IP converters 19A to 19C convert images transmitted via the network into formats unique to the corresponding monitors, and then output the resultant images. The input source-side IP converters 19 each function as an encoder. The image output-side IP converters 19 each function as a decoder. An input source includes a video source, for example.

The IP converters 19 may have various image processing functions. For example, the IP converters 19 may have functions for performing resolution conversion processing according to a destination to which an image is output, image angle correction and blurred image correction for an endoscopic image, object recognition processing, and the like.

These image processing functions may be unique to medical image devices connected to the IP converters 19 or may be upgradable externally. The image output-side (monitor-side) IP converters 19 may also perform processing such as synthesis of multiple images (e.g., PinP processing) and superimposition of annotation information.

The IP converters 19 have a protocol conversion function as a function of converting a received signal into a conversion signal conforming to a communication protocol that enables communications on a network such as the Internet, for example. The communication protocol to be set may be a given communication protocol. Furthermore, the IP converters 19 receive a digital signal as a signal that can be subjected to protocol conversion. Examples of the digital signal include an image signal and a pixel signal. The IP converters 19 may be incorporated in input source-side devices or image output-side devices.

The device group 13 belongs to, for example, an endoscopic surgery system. The device group 13 includes an endoscope, a display device for displaying an image imaged by the endoscope, and the like. On the other hand, the display devices 16A to 16D, the patient bed 17, and the light 18 are devices installed in the operating room separately from the endoscopic surgery system. These instruments for use in an operation or a diagnosis are each also called a medical instrument. The operating room server 11 and/or the IP SW 12 control/controls the behaviors of these medical instruments in cooperation. Furthermore, in a case where a surgical robot (surgical master-slave) system and a medical image capture device such as an X-ray machine are installed in the operating room, these instruments are also connected as the device group 13.

Here, of the devices of the operating room system 1, the device group 13, the ceiling camera 14, and the operative field camera 15 are devices each having a function of originating information to be displayed during an operation (hereinafter, also referred to as display information). Hereinafter, these devices are also referred to as originating source devices. Furthermore, the display devices 16A to 16D are devices to which the display information is output. Hereinafter, these devices are also referred to as output destination devices.

The operating room server 11 controls the processing in the operating room system 1 in a centralized manner.

The operating room server 11 has a function of controlling the behaviors of the originating source devices and output destination devices, acquiring the display information from the originating source devices, transmitting the display information to the output destination devices, and causing the output destination device to display or record the display information. The display information includes various images imaged during an operation, various kinds of information regarding the operation (e.g., physical information on a patient, results of past medical tests, and information about a technique), and the like.

Specifically, the operating room server 11 receives, as the display information from the device group 13, information about an image of a site in a patient's body cavity, the image being imaged by the endoscope. Furthermore, the operating room server 11 receives, as the display information from the ceiling camera 14, information about an image of the hands of the operator, the image being imaged by the ceiling camera 14. Furthermore, the operating room server 11 receives, as the display information from the operative field camera 15, information about an image of how things are at the entire operating room, the image being imaged by the operative field camera 15. In a case where the operating room system 1 includes other devices having an imaging function, the operating room server 11 may acquire, as display information from these devices, information about images imaged by these devices.

The operating room server 11 causes at least one of the display devices 16A to 16D as the output destination devices to display the acquired display information (i.e., the images imaged during the operation and the various kinds of information regarding the operation). In the example illustrated in FIG. 4, the display device 16A is a display device suspended from the ceiling of the operating room, and the display device 16B is a display device put on a wall surface of the operating room. The display device 16C is a display device mounted on a desk in the operating room, and the display device 16D is a mobile instrument (e.g., a tablet personal computer (PC) or a smartphone) having a display function.

As will be specifically described later, the operating room server 11 also performs, for example, processing for an operation video to be transmitted to the remote terminal 2.

The IP SW 12 is configured as one of input/output controllers for controlling input/output of image signals to/from the instruments connected thereto. For example, the IP SW 12 controls the input/output of the image signals on the basis of the control by the operating room server 11. The IP SW 12 controls high speed image signal transfer between the instruments disposed on the IP network.

The operating room system 1 may include devices placed outside the operating room. The devices outside the operating room are, for example, a server to be connected to a network constructed inside/outside the hospital, PCs to be used by medical staffs, a projector placed in a meeting room in the hospital, and the like. In the case where these external devices are located outside the hospital, the operating room server 11 may cause a display device in another hospital to display the display information via a video conference system or the like for telemedicine.

An external server 20 communicates with, for example, an in-hospital server outside the operating room, and the remote terminal 2. Image information on the interior of the operating room is transmitted to the remote terminal 2 via the external server 20. The data to be transmitted may be an operation video imaged by the endoscope or the like, metadata extracted from images, data indicating an operational status of an instrument to be connected, and the like.

In the operating room system 1, the IP network may be configured using a wired network. Alternatively, a part of the IP network or the entire IP network may be constructed using a wireless network. For example, the input source-side IP converters 19 having the wireless communication function may transmit received images to the image output-side IP converters 19 via a wireless communication network such as a fifth-generation mobile communication system (5G) or a sixth-generation mobile communication system (6G).

### <Functional Configuration of Operating Room Server 11>

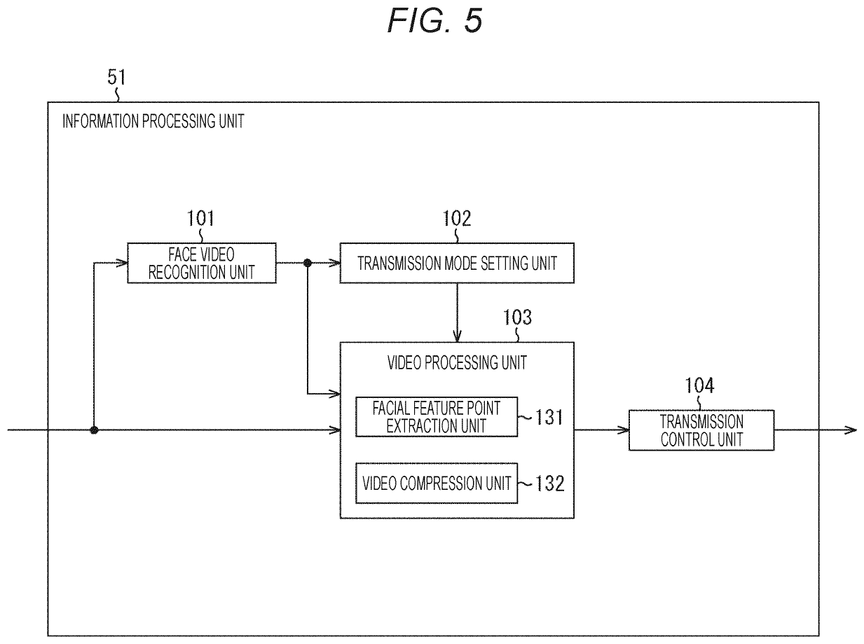

FIG. 5 is a block diagram illustrating an exemplary functional configuration of the operating room server 11 constituting a part of the operating room system 1.

As illustrated in FIG. 5, an information processing unit 51 is implemented in the operating room server 11. Each function illustrated in FIG. 5 is implemented in such a manner that a CPU of a computer constituting the operating room server 11 executes a predetermined program. The operating room server 11 is an information processing apparatus including the information processing unit 51.

The information processing unit 51 includes a face video recognition unit 101, a transmission mode setting unit 102, a video processing unit 103, and a transmission control unit 104. Operation videos imaged by the multiple cameras placed in the operating room are supplied to the face video recognition unit 101 and the video processing unit 103.

The face video recognition unit 101 determines whether or not each of the operation videos transmitted from the multiple cameras is a face video. As described above, the face video is an operation video possibly showing the patient's face.

### Face Video Determination Method 1

In a case where the IP converter 19 is connected to the instrument such as the camera, the IP converter 19 receives instrument data corresponding to information on the connected instrument, from the instrument. The instrument data received by the IP converter 19 is transmitted from the IP converter 19 to the operating room server 11, and then is supplied to the face video recognition unit 101.

The face video recognition unit 101 identifies an instrument (a camera) that has transmitted an operation video to be subjected to a determination, on the basis of the instrument data transmitted from the IP converter 19, and determines whether or not this operation video is a face video, in accordance with a type of the instrument that has transmitted the operation video, and the like. The instrument data includes information indicating the type of the instrument that has transmitted the operation video. For example, in a case where the instrument that has transmitted the operation video is the operative field camera 15 that images a range covering the patient bed 17, it is determined that the operation video to be subjected to a determination is a face video.

The determination whether or not the operation video is a face video may be made on the basis of metadata on the operation video. The metadata, such as that conforming to Digital Imaging and Communications in Medicine (DICOM), added to the operation video includes the information indicating the type of the instrument that has transmitted the operation video.

The IP converter 19 may add, as the metadata, a type of an instrument that images an operation video. Therefore, in a case where an instrument that has imaged a patient is known in advance, it is determined that an operation video transmitted from the relevant instrument is a face video.

The determination whether or not the operation video is a face video may be made using both the instrument data and the metadata added to the operation video. That is, the face video recognition unit 101 may determine whether or not the operation video is a face video, on the basis of at least one of the instrument data or the metadata added to the operation video.

### Face Video Determination Method 2

The face video recognition unit 101 analyzes the operation video, and determines whether or not the operation video of interest is a face video, on the basis of a result of the analysis. In this case, for example, face recognition is performed on each frame forming the operation video. In a case where the face is recognized, it is determined that the operation video of interest is a face video.

### Face Video Determination Method 3

The face video recognition unit 101 determines whether or not the operation video is a face video, on the basis of a result of selecting a video showing the patient's face. In this case, for example, a user such as the operator makes a selection as to which operation video is a video showing the patient's face. As described above, the determination whether or not the operation video is a face video may be made on the basis of the result of the selection by the user.

The information indicating the result of the determination by any of the foregoing methods is output from the face video recognition unit 101 to the transmission mode setting unit 102 and the video processing unit 103. The determination whether or not the operation video is a face video may be made by a combination of the multiple determination methods.

The transmission mode setting unit 102 identifies which one of the operation videos supplied from the IP converters 19 is a face video, on the basis of the result of the determination by the face video recognition unit 101. In a case where the user of the remote terminal 2 makes a request to transmit a face video, the transmission mode setting unit 102 sets the first transmission mode or the second transmission mode as a transmission mode for each face video.

For example, information indicating the content of a transmission setting is managed with the information associated with a terminal identification number of the remote terminal 2 used by the doctor at the remote location. As described above, the content of the transmission setting is prescribed by at least one of the state of the communication zone between the operating room server 11 and the remote terminal 2, the presence/absence of the patient's consent, or whether or not the remote terminal 2 is a hospital-aligned terminal.

### State of Communication Zone

The transmission mode setting unit 102 confirms a communication condition between the operating room server 11 and the remote terminal 2, and determines a state (wide/narrow) of the communication zone.

### Presence/Absence of Patient's Consent

Before conducting an operation, typically, a hospital asks patient's consent to transmit an operation video as personal information to the outside. The patient's consent about the handling of personal information is written in an electronic health record or the like and managed in a database. The transmission mode setting unit 102 is aligned with, for example, a device that manages the database (this device is not illustrated in FIG. 4), thereby referring to the electronic health record, and determines the presence/absence of the patient's consent.

For example, in a case where the patient's consent is obtained, the second transmission mode is set as appropriate in accordance with the state of the communication zone, whether or not the remote terminal 2 is a hospital-aligned terminal, and the like.

### Whether or not Remote Terminal 2 is Hospital-Aligned Terminal

The transmission mode setting unit 102 determines whether or not the remote terminal 2, to which the face video is transmitted, is a hospital-aligned terminal. In a case where the remote terminal 2, to which the face video is transmitted, is a hospital-aligned terminal, it is determined that the remote terminal 2 is a secure terminal (a terminal about which the security of a communication line is established).

For example, in a case where the terminal identification number of the remote terminal 2 is authenticated as a secure number or in a case where the remote terminal 2 is connected to the operating room server 11 via a dedicated line using a virtual private network (VPN), it is determined that the remote terminal 2 is a hospital-aligned terminal.

The transmission mode setting unit 102 sets the transmission mode as described with reference to FIG. 3 on the basis of the content of the transmission setting. The information on the transmission mode for each face video set by the transmission mode setting unit 102 is output to the video processing unit 103.

The video processing unit 103 includes a facial feature point extraction unit 131 and a video compression unit 132.

In a case where the first transmission mode is set as the transmission mode for the face video to be transmitted, the facial feature point extraction unit 131 analyzes the face video to extract a feature point of the patient's face.

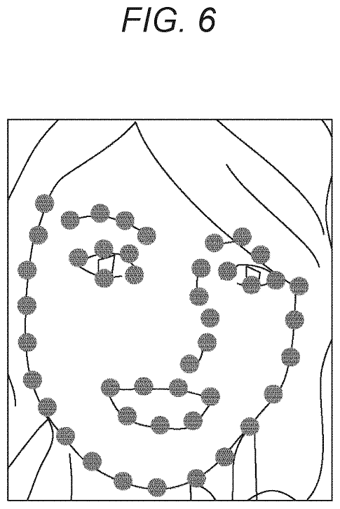

FIG. 6 is a diagram illustrating an exemplary feature point extracted from the face by the facial feature point extraction unit 131.

As illustrated in FIG. 6, a feature point to be extracted is information on a position of each site of the patient's face, information on the contour of the face (contour data), or the like. In FIG. 6, a colored small circle indicates a position where a feature point is extracted. In a case where there is a possibility that an individual is identified from a feature point of the contour of a patient's face to be used as it is, the feature point may be replaced with a feature point of a standard face, and the feature point of the standard face may be transmitted to the remote terminal 2.

The information on the feature point extracted by analyzing the face video is output to the transmission control unit 104. A category of an expression classified on the basis of the feature extracted by analyzing the face video may be output as the feature point of the face to the transmission control unit 104. Examples of the category of the expression include an expression on rest and quiet, a slightly painful expression, a very painful expression, and the like.

A degree of anguish may be calculated on the basis of the feature of the face, and the category of the expression may be classified on the basis of the calculated degree of anguish. Furthermore, the degree of anguish calculated on the basis of the feature may be output as the feature point of the face to the transmission control unit 104.

Referring back to FIG. 5, in a case where the second transmission mode is set as the transmission mode for the face video to be transmitted, the video compression unit 132 compresses the face video.

An operation video to be transmitted to the operating room server 11 is, for example, a video having a large data amount, such as a 4K video or a RAW video. In a case where the second transmission mode is set as the transmission mode for the face video, for example, compression processing is performed to reduce the 4K-resolution face video to the FHD-resolution face video.

The face video compressed by the video compression unit 132 is output to the transmission control unit 104. Also in a case where it is determined that the operation video to be transmitted is different from a face video, the compression processing is appropriately performed on the operation video by the video compression unit 132, and the compressed operation video is output to the transmission control unit 104.

In the case where the first transmission mode is set as the transmission mode for the face video to be transmitted, the transmission control unit 104 transmits to the remote terminal 2 the information on the feature point of the face supplied from the facial feature point extraction unit 131.

Furthermore, in the case where the second transmission mode is set as the transmission mode for the face video to be transmitted, the transmission control unit 104 transmits to the remote terminal 2 the compressed face video supplied from the video compression unit 132.

### <Behavior of Operating Room Server 11>

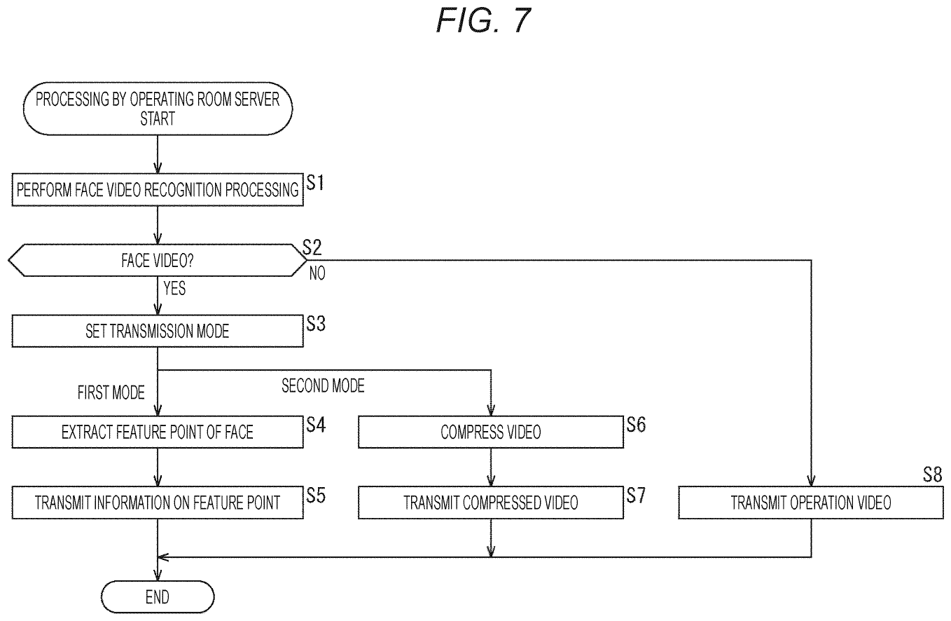

With reference to a flowchart of FIG. 7, a description will be given of processing to be performed by the operating room server 11 having the foregoing configuration. The processing illustrated in FIG. 7 starts, for example, when the user of the remote terminal 2 selects an operation video to be transmitted.

In step S1, the face video recognition unit 101 performs face video recognition processing on an operation video imaged by each of the cameras.

In step S2, the face video recognition unit 101 determines whether or not one of the operation videos selected as a transmission target is a face video.

In a case where it is determined in step S2 that the selected operation video is a face video, next, in step S3, the transmission mode setting unit 102 identifies the state of the communication line, and the like, and sets the transmission mode for the face video to be transmitted.

In a case where the first transmission mode is set in step S3, next, in step S4, the facial feature point extraction unit 131 analyzes the face video to extract a feature point of the patient's face.

In step S5, the transmission control unit 104 transmits the information on the feature point of the face to the remote terminal 2. The remote terminal 2 performs, for example, display of an avatar image representing the patient's expression as described above, on the basis of the information on the feature point of the face.

On the other hand, in a case where the second transmission mode is set in step S3, next, in step S6, the video compression unit 132 compresses the face video.

In step S7, the transmission control unit 104 transmits the compressed face video to the remote terminal 2. The remote terminal 2 displays the video showing the patient's face, on the basis of the face video obtained by decompression.

Also in a case where it is determined in step S2 that the operation video is different from a face video, next, in step S8, the transmission control unit 104 transmits to the remote terminal 2 the operation video compressed appropriately.

The transmission of the information on the feature point in step S5 and the transmission of the operation video in steps S7 and S8 are continuously carried out during the transmission of the face video. For example, in a case where the user of the remote terminal 2 selects an end of the transmission of the operation video, the processing illustrated in FIG. 7 ends.

Through the foregoing processing, the operating room server 11 is capable of appropriately selecting the mode for transmitting the information regarding the patient's expression.

### <<Configuration and Behavior of Remote Terminal>>

### <Functional Configuration of Remote Terminal 2>

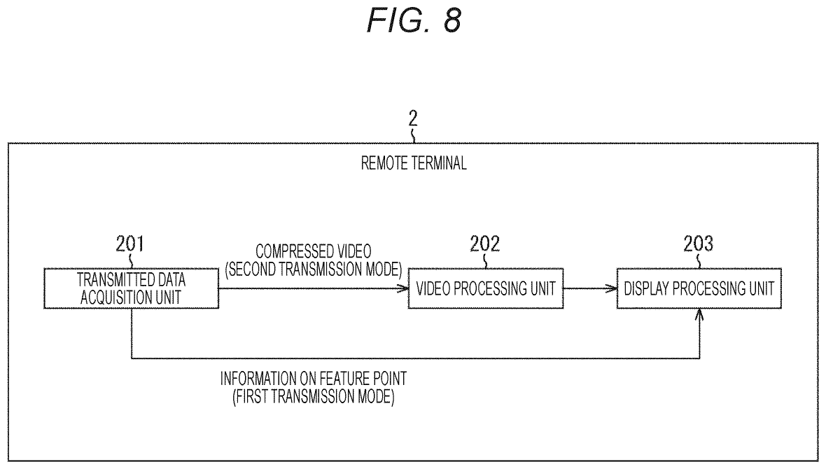

FIG. 8 is a block diagram illustrating an exemplary functional configuration of the remote terminal 2. Each function illustrated in FIG. 8 is implemented in such a manner that a CPU of a computer constituting the remote terminal 2 executes a predetermined program.

In the remote terminal 2, a transmitted data acquisition unit 201, a video processing unit 202, and a display control unit 203 are implemented.

The transmitted data acquisition unit 201 controls a communication module provided in the remote terminal 2 to acquire information transmitted from the operating room server 11.

For example, in the case where the first transmission mode is set as the transmission mode for the face video, the transmitted data acquisition unit 201 acquires the information on the feature point of the face transmitted from the operating room server 11. Furthermore, in the case where the second transmission mode is set as the transmission mode for the face video, the transmitted data acquisition unit 201 acquires the compressed face video transmitted from the operating room server 11.

The transmitted data acquisition unit 201 outputs the information on the feature point of the face to the display control unit 203, and outputs the compressed face video to the video processing unit 202.

In a case where the operation video transmitted from the operating room server 11 is different from a face video, the transmitted data acquisition unit 201 outputs the acquired operation video to the video processing unit 202. The case where the operation video transmitted from the operating room server 11 is a face video is mainly described here. In a case where the operation video different from a face video is compressed, the compressed operation video is appropriately subjected to processing similar to the processing for the compressed face video.

The video processing unit 202 performs decompression processing on the face video supplied from the transmitted data acquisition unit 201. Decompression of a high-resolution face video is performed using, for example, an inference model generated by machine learning. In this case, an inference model constituted of a neural network or the like to which a low-resolution face video is input and from which a high-resolution face video is output is prepared for the video processing unit 202 in advance. Furthermore, the decompression of the high-resolution face video may be performed using super-resolution processing.

Therefore, in a case where the face video is transmitted to the remote terminal 2, it is possible to transmit the face video at a high compression rate. The face video decompressed by the video processing unit 202 is output to the display control unit 203.

The display control unit 203 causes the display to display an avatar image showing a changed expression, on the basis of the information on the feature point of the face supplied from the transmitted data acquisition unit 201. Furthermore, in a case where an image of the patient's face is provided, the display control unit 203 changes the image of the patient's face in accordance with the information on the feature point of the face, and causes the display to display the changed image of the patient's face.

Furthermore, the display control unit 203 causes the display to display the decompressed high-resolution face video supplied from the video processing unit 202.

### <Behavior of Remote Terminal 2>

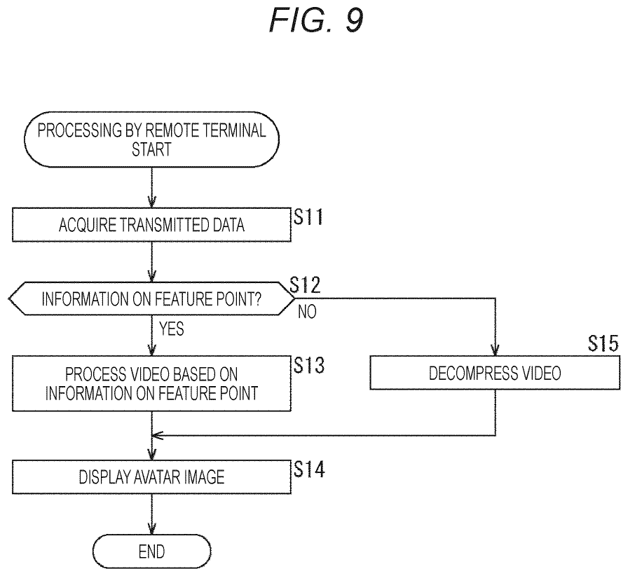

With reference to a flowchart of FIG. 9, a description will be given of processing to be performed by the remote terminal 2 having the foregoing configuration. The processing illustrated in FIG. 9 starts, for example, when data is transmitted from the operating room server 11.

In step S11, the transmitted data acquisition unit 201 acquires transmitted data that is transmitted from the operating room server 11.

In step S12, the transmitted data acquisition unit 201 determines whether or not the data transmitted from the operating room server 11 is the information on the feature point of the patient's face.

In a case where it is determined in step S12 that the data transmitted from the operating room server 11 is the information on the feature point of the patient's face, next, in step S13, the display control unit 203 performs video processing for performing display of an avatar image, and the like, on the basis of the information on the feature point of the face.

In step S14, the display control unit 203 causes the display to display the avatar image generated by the video processing, and the like.

On the other hand, in a case where it is determined in step S12 that the data transmitted from the operating room server 11 is different from the information on the feature point of the patient's face, next, in step S15, the video processing unit 202 performs decompression processing on the face video supplied from the transmitted data acquisition unit 201. After the processing in step S15, the processing then proceeds to step S14. In step S14, the high-resolution face video obtained by the decompression processing is displayed on the display.

The series of the processing described above is continuously performed until, for example, the user of the remote terminal 2 selects an end of the transmission of the operation video.

The foregoing processing allows the user of the remote terminal 2 at the remote location to confirm a change in the patient's face and provide instructions in real-time to the operator and the like in the operating room, while viewing the video displayed on the display.

### Modifications

### <Second Transmission Mode>

In the case where the first transmission mode is set as the transmission mode for the face video, when the user of the remote terminal 2 makes a request to transmit a video showing an actual patient's face, the transmission mode may be switched from the first transmission mode to the second transmission mode in accordance with the request from the user.

In this case, the transmission mode setting unit 102 determines the state of the communication zone, the presence/absence of the patient's consent, and the like as described above. In a case where the state of the communication zone is wide and the patient's consent is obtained, the transmission mode setting unit 102 switches the transmission mode for the face video from the first transmission mode to the second transmission mode to start the transmission of the compressed face video.

As described above, in the case where transmission of the personal information is permitted and the state of the communication zone is wide, the operating room server 11 transmits the face video to the remote terminal 2. Since the face video rather than the information on the feature point of the patient's face is transmitted, the doctor who is at the remote location is able to more accurately grasp the patient's expression.

In a case where the first transmission mode is set as the transmission mode for the face video and the information on the degree of anguish is transmitted as the information on the feature point of the face to the remote terminal 2, the transmission mode may be switched from the first transmission mode to the second transmission mode when the degree of anguish is more than a certain threshold value.

As described above, the transmission of the face video starts when the degree of anguish is more than the certain threshold value, so that the user of the remote terminal 2 is able to more quickly notice a change in the patient's condition.

A third transmission mode may be provided, which is a transmission mode for transmitting to the remote terminal 2 neither the information on the feature point of the face nor the compressed face video. For example, in a case where the degree of anguish is less than the certain threshold value, the transmission mode is switched from the first transmission mode or the second transmission mode to the third transmission mode.

Switching to the third transmission mode enables a reduction in data communication amount.

In a case where a request is sent from the remote terminal 2, a compression rate and a frame rate may be changed in accordance with the state of the communication zone, and the like.

<Example in which Another Instrument Makes Face Video Determination>

The face video determination may be made by another instrument rather than the operating room server 11.

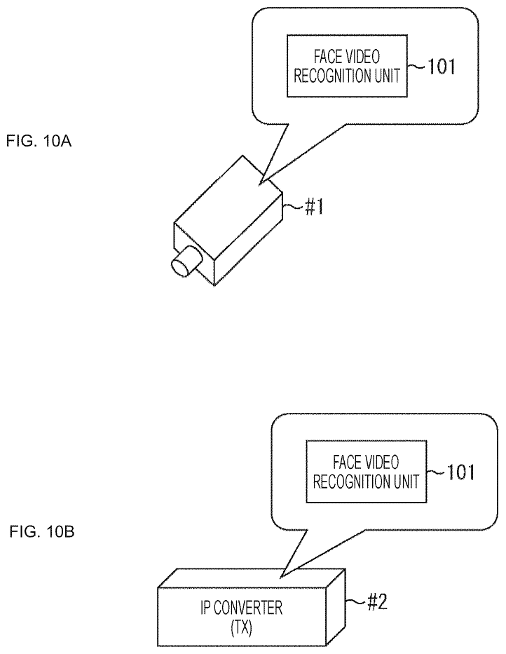

FIG. 10 is a diagram illustrating exemplary another instrument that makes a face video determination.

As illustrated in A of FIG. 10, the camera #1 may make a face video determination. In this case, the face video recognition unit 101 is implemented by the camera #1.

As illustrated in B of FIG. 10, the IP converter #2 may make a face video determination. In this case, the face video recognition unit 101 is implemented by the IP converter #2.

The face video recognition unit 101 provided in the camera #1 or the IP inverter #2 makes a determination whether or not an operation video is a face video, in a manner similar to that of the face video recognition unit 101 (FIG. 5) of the operating room server 11.

<Example in which Another Instrument Performs Operation Video Processing>

The operation video processing may be performed by another instrument rather than the operating room server 11.

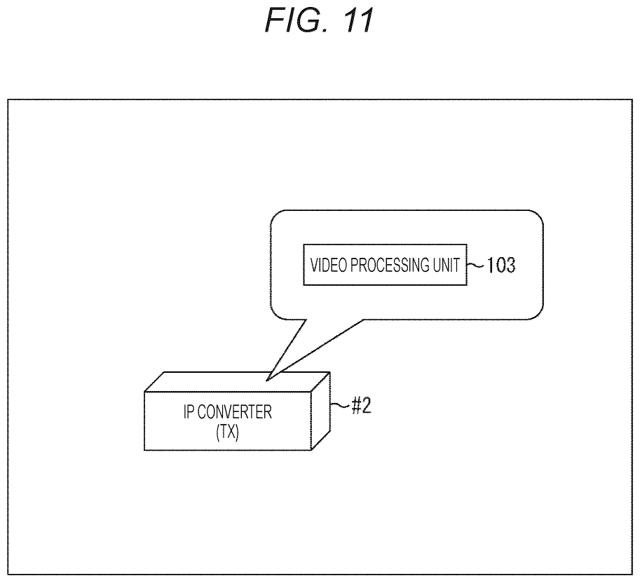

FIG. 11 is a diagram illustrating an exemplary IP converter #2 that performs operation video processing.

As illustrated in FIG. 11, in a case where the IP converter #2 performs the operation video processing, the video processing unit 103 is implemented by the IP converter #2. The video processing unit 103 provided in the IP inverter #2 performs processing on an operation video, in a manner similar to that of the video processing unit 103 (FIG. 5) of the operating room server 11.

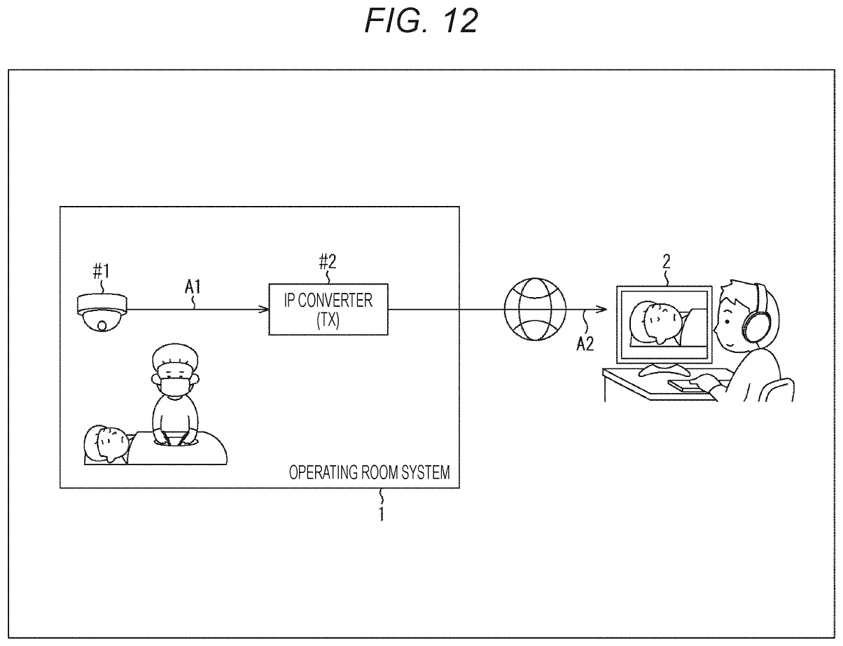

In this case, as illustrated in FIG. 12, the information on the feature point of the face or the compressed face video may be directly transmitted from the IP converter #2 to the remote terminal 2 without via the operating room server 11.

The IP converter #2 illustrated in FIG. 12 performs video processing such as facial feature point extraction and compression, on the face video supplied from the camera #1. The IP converter #2 subjects transmission data obtained through the video processing, to IP conversion, and transmits the resultant transmission data to the remote terminal 2.

As described above, at least one of the functional units constituting the information processing unit 51 (FIG. 5) can be implemented in an instrument different from the operating room server 11. At least one of the functional units constituting the information processing unit 51 may be implemented in another device such as a server on a cloud.

### <Facial Feature Point Extraction>

In a case where it is difficult to extract the feature point of the face since the patient's face is covered with something, a notification about detection of an unusual state may be provided to the operator in the operating room and the user of the remote terminal 2.

In this case, for example, the face video recognition unit 101 of the operating room server 11 measures a degree of reliability as to the extraction of the feature point of the face. In a case where the measured degree of reliability is less than a certain threshold value, an alert representing detection of an unusual state (warning information) is transmitted together with the information on the feature point of the face to, for example, the remote terminal 2. The alert to be transmitted to the remote terminal 2 is an alert indicating that the feature point of the face is inaccurate depending on circumstances.

An alert indicating that the feature point of the face cannot be extracted may be transmitted without transmitting the information on the feature point of the face.

An alert that is generated for recommending that the transmission mode is switched from the first transmission mode to the second transmission mode may be transmitted to the remote terminal 2.

A cycle of the facial feature point extraction is not necessarily fixed, and may be changed in accordance with a change in the feature point of the patient's face. For example, in a case where the degree of patient's anguish is large, the extraction cycle is shortened, and feature points are extracted from all frames. The value of the extraction cycle may be changeable in accordance with the urgency of the operation and the state of the communication zone.

In a case where a feature point of a face is extracted using an image sensor in a camera, the power consumption by the camera may be reduced in such a manner that a face video is output when a change in the feature point is large. For example, in a case where a degree of anguish based on the feature point of the face is calculated by the image sensor, the information on the feature point of the face or the face video is output from the camera only when the degree of anguish is more than a certain threshold value. The frame rate of the face video may be set high only when the degree of anguish is more than the certain threshold value.

An operation video may be stored in a given server in addition to a result of analysis on the change in the feature point of the face. Therefore, it is possible to replace the face video with an avatar image even after the operation and to use the operation video again while keeping patient's anonymity.

### Others

The foregoing description concerns the case where the information processing system illustrated in FIG. 1 is used for a remote operation in a medical situation. The present technology is also applicable to a case of transmitting a video different from an operation video to the remote terminal 2 that is at a remote location. For example, the present technology is also applicable to a case of transmitting a face video imaged by a security camera placed in a district area and a face video imaged by a camera placed in a live event venue.

Information regarding a person's expression shown in a face video is not necessarily presented through display on a screen, and may be presented through other transmission means such as output of voice and light emission from an LED.

### Program

The foregoing series of processing may be performed by hardware or may be performed by software. In a case where the series of processing is performed by software, a program that constitutes the software is installed in a computer incorporated in special-purpose hardware, a general-purpose personal computer, or the like through a program recording medium.

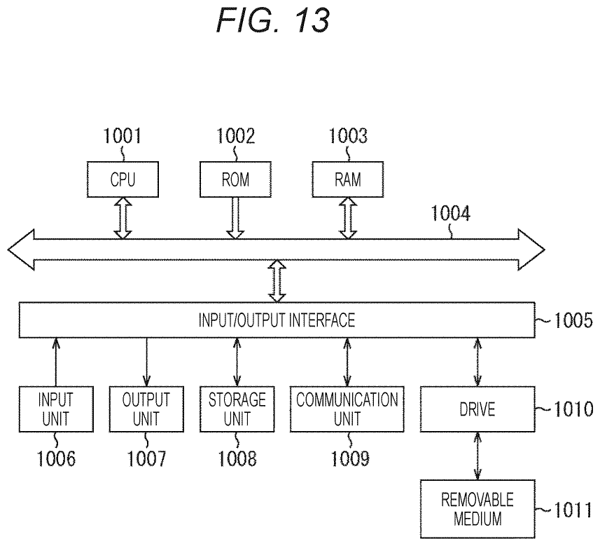

FIG. 13 is a block diagram illustrating an exemplary hardware configuration of a computer that executes the foregoing series of processing by a program. The remote terminal 2 and the operating room server 11 each have a configuration similar to the configuration illustrated in FIG. 13.

A central processing unit (CPU) 1001, a read-only memory (ROM) 1002, and a random access memory (RAM) 1003 are connected to each other via a bus 1004.

An input/output interface 1005 is also connected to the bus 1004. An input unit 1006, an output unit 1007, a storage unit 1008, a communication unit 1009, and a drive 1010 are connected to the input/output interface 1005. The drive 1010 drives a removable medium 1011 such as a magnetic disk, an optical disk, a magneto-optical disk, or a semiconductor memory.

The computer configured as described above performs the foregoing series of processing in such a manner that, for example, the CPU 1001 loads a program stored in the storage unit 1008 onto the RAM 1003 via the input/output interface 1005 and the bus 1004, and then executes the program.

The program to be executed by the CPU 1001 is recorded in the removable medium 1011 or is provided via a wired or wireless transmission medium such as a local area network, the Internet, or digital broadcast, and then is installed in the storage unit 1008, for example.

Note that the program to be executed by the computer may be a program by which processing is performed in a time-series manner in accordance with a sequence described herein, or may be a program by which processing is performed in parallel or at a required timing such as a timing when the program is invoked.

The advantageous effects described herein are merely exemplary. The present technology is not limited to these advantageous effects and may produce other advantageous effects.

Embodiments of the present technology are not limited to the foregoing embodiments and may be modified variously without departing from the scope of the present technology.

For example, the present technology may adopt a configuration of cloud computing in which one function is shared between a plurality of devices via a network such that the devices process this function in cooperation with each other.

Furthermore, each step described in the foregoing flowcharts may be carried out by a single device or may be carried out by a plurality of devices in a shared manner.

In addition, in a case where one step includes multiple processes, the multiple processes in the one step may be performed by a single device or may be performed by a plurality of devices in a shared manner.

### Exemplary Combinations of Configurations

The present technology may adopt the following configurations.

(1)

An information processing apparatus including:
a transmission mode setting unit that sets, in a case where an operation video requested from a remote terminal is a face video possibly showing a patient's face, a transmission mode on the basis of a transmission setting defined for the remote terminal to which the face video is transmitted; and
a transmission control unit that transmits information on a feature point of the patient's face extracted from the face video to the remote terminal in a case where a first transmission mode is set, and transmits the compressed face video to the remote terminal in a case where a second transmission mode is set.
   (2)

The information processing apparatus as recited in (1), further including
a face video recognition unit that determines whether or not the operation video shows the patient's face.
   (3)

The information processing apparatus as recited in (1) or (2), in which
the transmission setting is prescribed by at least one of a state of a communication zone, presence/absence of patient's consent, or whether or not the remote terminal is a hospital-aligned terminal.
   (4)

The information processing apparatus as recited in any one of (1) to (3), further including
a video processing unit that extracts the feature point from the face video in the case where the first transmission mode is set, and compresses the face video in the case where the second transmission mode is set.
   (5)

The information processing apparatus as recited in (4), in which
the video processing unit extracts, as the feature point, contour data on the patient's face.
   (6)

The information processing apparatus as recited in (4), in which
the video processing unit extracts, as the feature point, a category of an expression classified on the basis of a feature of the patient's face.
   (7)

The information processing apparatus as recited in (6), in which
the video processing unit classifies the category of the expression on the basis of a degree of anguish according to the feature of the patient's face.
   (8)

The information processing apparatus as recited in any one of (1) to (7), in which
the transmission mode setting unit switches the transmission mode from the first transmission mode to the second transmission mode on the basis of a request from the remote terminal.
   (9)

The information processing apparatus as recited in any one of (2) to (8), in which
the face video recognition unit determines whether or not the operation video shows the patient's face, on the basis of at least one of information on an instrument configured to image the operation video or metadata added to the face video.
   (10)

The information processing apparatus as recited in any one of (2) to (8), in which
the face video recognition unit determines whether or not the operation video shows the patient's face, on the basis of a result of analysis on the operation video.
   (11)

The information processing apparatus as recited in any one of (2) to (8), in which
the face video recognition unit determines whether or not the operation video shows the patient's face, on the basis of a result of selection by a user.
   (12)

An information processing method including:
setting, by an information processing apparatus, in a case where an operation video requested from a remote terminal is a face video possibly showing a patient's face, a transmission mode on the basis of a transmission setting defined for the remote terminal to which the face video is transmitted;
transmitting, by the information processing apparatus, information on a feature point of the patient's face extracted from the face video to the remote terminal in a case where a first transmission mode is set; and
transmitting, by the information processing apparatus, the compressed face video to the remote terminal in a case where a second transmission mode is set.
   (13)

A program for causing a computer to execute processing,
the processing including:
setting, in a case where an operation video requested from a remote terminal is a face video possibly showing a patient's face, a transmission mode on the basis of a transmission setting defined for the remote terminal to which the face video is transmitted;
transmitting information on a feature point of the patient's face extracted from the face video to the remote terminal in a case where a first transmission mode is set; and
transmitting the compressed face video to the remote terminal in a case where a second transmission mode is set.
   (14)

An information processing terminal including:
a transmitted data acquisition unit that sets, in a case where an operation video of which transmission is requested is a face video possibly showing a patient's face, a transmission mode on the basis of a transmission setting defined for a destination to which the face video is transmitted, transmits information on a feature point of the patient's face extracted from the face video in a case where a first transmission mode is set, and acquires information transmitted from an information processing apparatus configured to transmit the compressed face video, in a case where a second transmission mode is set; and
a display control unit that displays information regarding a patient's expression on the basis of the acquired information.
   (15)

The information processing terminal as recited in (14), in which
the display control unit changes display of an avatar image on the basis of the information on the feature point of the patient's face acquired in the case where the first transmission mode is set.
   (16)

The information processing terminal as recited in (14), in which
the display control unit changes an image of the patient's face on the basis of the information on the feature point of the patient's face acquired in the case where the first transmission mode is set.
   (17)

The information processing terminal as recited in any one of (14) to (16), further including
a video processing unit that decompresses the compressed face video acquired in the case where the second transmission mode is set,
in which
the display control unit displays the decompressed face video as the information regarding the patient's expression.
   (18)

An information processing method including:
setting, by an information processing terminal, in a case where an operation video of which transmission is requested is a face video possibly showing a patient's face, a transmission mode on the basis of a transmission setting defined for a destination to which the face video is transmitted, transmitting, by the information processing terminal, information on a feature point of the patient's face extracted from the face video in a case where a first transmission mode is set, and acquiring, by the information processing terminal, information transmitted from an information processing apparatus configured to transmit the compressed face video, in a case where a second transmission mode is set; and
displaying, by the information processing terminal, information regarding a patient's expression on the basis of the acquired information.
   (19)

A program for causing a computer to execute processing,
the processing including:
setting, in a case where an operation video of which transmission is requested is a face video possibly showing a patient's face, a transmission mode on the basis of a transmission setting defined for a destination to which the face video is transmitted, transmitting information on a feature point of the patient's face extracted from the face video in a case where a first transmission mode is set, and acquiring information transmitted from an information processing apparatus configured to transmit the compressed face video, in a case where a second transmission mode is set; and
displaying information regarding a patient's expression on the basis of the acquired information.
   (20)

An information processing apparatus including:
a transmission mode setting unit that sets, in a case where a video requested from a remote terminal is a face video possibly showing a person's face, a transmission mode on the basis of a transmission setting defined for the remote terminal to which the face video is transmitted; and
a transmission control unit that transmits information on a feature point of the person's face extracted from the face video to the remote terminal in a case where a first transmission mode is set, and transmits the compressed face video to the remote terminal in a case where a second transmission mode is set.

### REFERENCE SIGNS LIST

1 Operating room system
2 Remote terminal
11 Operating room server
51 Information processing unit
101 Face video recognition unit
102 Transmission mode setting unit
103 Video processing unit
104 Transmission control unit
131 Facial feature point extraction unit
132 Video compression unit
201 Transmitted data acquisition unit
202 Video processing unit
203 Display control unit

1. An information processing apparatus comprising:
a transmission mode setting unit that sets, in a case where an operation video requested from a remote terminal is a face video possibly showing a patient's face, a transmission mode on a basis of a transmission setting defined for the remote terminal to which the face video is transmitted; and
a transmission control unit that transmits information on a feature point of the patient's face extracted from the face video to the remote terminal in a case where a first transmission mode is set, and transmits the compressed face video to the remote terminal in a case where a second transmission mode is set.

2. The information processing apparatus according to claim 1, further comprising
a face video recognition unit that determines whether or not the operation video shows the patient's face.

3. The information processing apparatus according to claim 1, wherein
the transmission setting is prescribed by at least one of a state of a communication zone, presence/absence of patient's consent, or whether or not the remote terminal is a hospital-aligned terminal.

4. The information processing apparatus according to claim 1, further comprising
a video processing unit that extracts the feature point from the face video in the case where the first transmission mode is set, and compresses the face video in the case where the second transmission mode is set.

5. The information processing apparatus according to claim 4, wherein
the video processing unit extracts, as the feature point, contour data on the patient's face.

6. The information processing apparatus according to claim 4, wherein
the video processing unit extracts, as the feature point, a category of an expression classified on a basis of a feature of the patient's face.

7. The information processing apparatus according to claim 6, wherein
the video processing unit classifies the category of the expression on a basis of a degree of anguish according to the feature of the patient's face.

8. The information processing apparatus according to claim 1, wherein
the transmission mode setting unit switches the transmission mode from the first transmission mode to the second transmission mode on a basis of a request from the remote terminal.

9. The information processing apparatus according to claim 2, wherein
the face video recognition unit determines whether or not the operation video shows the patient's face, on a basis of at least one of information on an instrument configured to image the operation video or metadata added to the face video.

10. The information processing apparatus according to claim 2, wherein
the face video recognition unit determines whether or not the operation video shows the patient's face, on a basis of a result of analysis on the operation video.

11. The information processing apparatus according to claim 2, wherein
the face video recognition unit determines whether or not the operation video shows the patient's face, on a basis of a result of selection by a user.

12. An information processing method comprising:
setting, by an information processing apparatus, in a case where an operation video requested from a remote terminal is a face video possibly showing a patient's face, a transmission mode on a basis of a transmission setting defined for the remote terminal to which the face video is transmitted;
transmitting, by the information processing apparatus, information on a feature point of the patient's face extracted from the face video to the remote terminal in a case where a first transmission mode is set; and
transmitting, by the information processing apparatus, the compressed face video to the remote terminal in a case where a second transmission mode is set.

13. A program for causing a computer to execute processing,
the processing comprising:
setting, in a case where an operation video requested from a remote terminal is a face video possibly showing a patient's face, a transmission mode on a basis of a transmission setting defined for the remote terminal to which the face video is transmitted;
transmitting information on a feature point of the patient's face extracted from the face video to the remote terminal in a case where a first transmission mode is set; and
transmitting the compressed face video to the remote terminal in a case where a second transmission mode is set.

14. An information processing terminal comprising:
a transmitted data acquisition unit that sets, in a case where an operation video of which transmission is requested is a face video possibly showing a patient's face, a transmission mode on a basis of a transmission setting defined for a destination to which the face video is transmitted, transmits information on a feature point of the patient's face extracted from the face video in a case where a first transmission mode is set, and acquires information transmitted from an information processing apparatus configured to transmit the compressed face video, in a case where a second transmission mode is set; and
a display control unit that displays information regarding a patient's expression on a basis of the acquired information.

15. The information processing terminal according to claim 14, wherein
the display control unit changes display of an avatar image on a basis of the information on the feature point of the patient's face acquired in the case where the first transmission mode is set.

16. The information processing terminal according to claim 14, wherein
the display control unit changes an image of the patient's face on a basis of the information on the feature point of the patient's face acquired in the case where the first transmission mode is set.

17. The information processing terminal according to claim 14, further comprising
a video processing unit that decompresses the compressed face video acquired in the case where the second transmission mode is set,
wherein
the display control unit displays the decompressed face video as the information regarding the patient's expression.

18. An information processing method comprising:
setting, by an information processing terminal, in a case where an operation video of which transmission is requested is a face video possibly showing a patient's face, a transmission mode on a basis of a transmission setting defined for a destination to which the face video is transmitted, transmitting, by the information processing terminal, information on a feature point of the patient's face extracted from the face video in a case where a first transmission mode is set, and acquiring, by the information processing terminal, information transmitted from an information processing apparatus configured to transmit the compressed face video, in a case where a second transmission mode is set; and
displaying, by the information processing terminal, information regarding a patient's expression on a basis of the acquired information.

19. A program for causing a computer to execute processing,
the processing comprising:
setting, in a case where an operation video of which transmission is requested is a face video possibly showing a patient's face, a transmission mode on a basis of a transmission setting defined for a destination to which the face video is transmitted, transmitting information on a feature point of the patient's face extracted from the face video in a case where a first transmission mode is set, and acquiring information transmitted from an information processing apparatus configured to transmit the compressed face video, in a case where a second transmission mode is set; and
displaying information regarding a patient's expression on a basis of the acquired information.

20. An information processing apparatus comprising:
a transmission mode setting unit that sets, in a case where a video requested from a remote terminal is a face video possibly showing a person's face, a transmission mode on a basis of a transmission setting defined for the remote terminal to which the face video is transmitted; and
a transmission control unit that transmits information on a feature point of the person's face extracted from the face video to the remote terminal in a case where a first transmission mode is set, and transmits the compressed face video to the remote terminal in a case where a second transmission mode is set.
